(19) United States
(12) Patent Application Publication
(10) Pub. No.: US 2017/0327906 A1
(43) Pub. Date: Nov. 16, 2017

(54) METHODS FOR PREDICTING AND REDUCING RISK OF COPPER DEFICIENCY IN A RUMINANT SUBJECT OR A RUMINANT HERD

(71) Applicants: Vanderbilt University, Nashville, TN (US); Middle Tennessee State University, Murfreesboro, TN (US)

(72) Inventors: James West, Nashville, TN (US); Warren Gill, Murfreesboro, TN (US); Jessica Carter, Murfreesboro, TN (US)

(21) Appl. No.: 15/595,777

(22) Filed: May 15, 2017

Related U.S. Application Data
(60) Provisional application No. 62/335,752, filed on May 13, 2016.

(51) Int. Cl. C12Q 1/68; G01N 33/84

(52) U.S. Cl. CPC C12Q 1/6895; G01N 33/84; C12Q 1/6883; C12Q 2600/158

(57) ABSTRACT

A method for treating copper deficiency in a ruminant subject or a ruminant herd is dexcribed. In some embodiments, the method involves providing a sample from the subject or at least one subject in the herd; measuring an amount of (i) fungal infection in the sample; (ii) expression of tyrosinase (Tyr) in the sample, and/or (iii) expression of tyrosinase-related protein (Tyrp) in the sample; (c) estimating the level of copper deficiency in the subject or herd by correlating the measured amount to a reference; (d) calibrating a predicted effective copper supplementation dose based on the estimated level of coppery deficiency in the subject or herd; and (e) administering the predicted effective copper supplementation dose to the subject or at least one subject in the herd and/or inoculating the subject or at least one subject in the herd with a fungus or fungal isolate.

### RELATED APPLICATIONS

This application claims priority from U.S. Provisional Application Ser. No. 62/335,752 filed May 13, 2016, the entire disclosure of which is incorporated herein by this reference.

### TECHNICAL FIELD

The presently-disclosed subject matter relates to prediction of coppery deficiency, including early prediction of copper deficiency in a ruminant subject, and to reducing risk of copper deficiency and morbidity and mortality associated with copper deficiency while avoiding copper metal poisoning.

### INTRODUCTION

Copper deficiency is a serious problem for grazing ruminants. It affects nearly one third of all beef cattle in the United States, causing significant morbidity and mortality, and resulting in a substantial economic impact in cattle production. Treatment of copper deficiency consists of copper supplementation in diet; however, excess copper is toxic, resulting in metal poisoning. Thus, copper supplementation should be calibrated to levels of copper deficiency. Unfortunately, blood levels of copper do not correlate with copper deficiency, and a liver biopsy is the current gold-standard for determining copper deficiency:

Accordingly, there remains a need in the art for a technology which provides a non-invasive or low-invasive prediction of copper deficiency and reduction of the risk of copper deficiency and conditions associated therewith, while also limiting the risk of metal poisoning.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method for treating copper deficiency in a ruminant subject or a ruminant herd. In some embodiments, the method involves (a) providing a sample from the subject or at least one subject in the herd; (b) measuring an amount of (i) fungal infection in the sample; (ii) expression of tyrosinase (Tyr) in the sample, and/or (iii) expression of tyrosinase-related protein (Tyrp) in the sample; (c) estimating the level of copper deficiency in the subject or herd by correlating the measured amount to a reference; (d) calibrating a predicted effective copper supplementation dose based on the estimated level of coppery deficiency in the subject or herd; and (e) administering the predicted effective copper supplementation dose to the subject or at least one subject in the herd and/or inoculating the subject or at least one subject in the herd with a fungus or fungal isolate.

The presently-disclosed subject matter includes a method for predicting copper deficiency in and treating copper deficiency in a ruminant subject or a ruminant herd. In some embodiments, the method involves (a) receiving a dataset, the dataset comprising measured amounts of (i) fungal infection in a sample from the subject or at least one subject in the herd, (ii) expression of tyrosinase (Tyr) in the sample, and/or (iii) expression of tyrosinase-related protein (Tyrp) in the sample; and optionally further comprising measured amounts of (iv)selenium, cobalt, and/or zinc in the sample; and (b) processing the dataset, where processing comprises (i) calculating a risk index, wherein the calculating comprises: applying predetermined weights to the measured amounts; and solving one or more nonlinear differential equations, wherein the one or more nonlinear differential equations are based, at least in part, on the weights for the measured amounts; and (ii) correlating a risk index to an odds ratio, wherein the correlating comprises solving a logit probability model.

The presently-disclosed subject matter includes a system for predicting copper deficiency in a ruminant subject or a ruminant herd, which involves (a) a data access device configured to receive measured amounts of (i) fungal infection in a sample from the subject or at least one subject in the herd, (ii) expression of tyrosinase (Tyr) in the sample, and/or (iii) expression of tyrosinase-related protein (Tyrp) in the sample; and optionally measured amounts of (iv) selenium, cobalt, and/or zinc in the sample; and (b) a server in data communication with the data access device configured to perform: (i) calculating a risk index, wherein the calculating comprises: applying a predetermined weight to the measured amounts; and solving one or more nonlinear differential equations, wherein the one or more nonlinear differential equations is based, at least in part, on the weights for the measured amounts; and (ii) correlating the risk index to an odds ratio, wherein the correlating comprises solving a logit probability model.

The presently-disclosed subject matter includes a method for identifying fungal infection and expression of tyrosinase and tyrosinase-related protein in a ruminant subject or a ruminant herd. In some embodiments, the method involves (a) providing a sample from the subject or at least one subject in the herd; (b) measuring an amount of (i) fungal infection in the sample; (ii) expression of tyrosinase (Tyr) in the sample, and/or (iii) expression of tyrosinase-related protein (Tyrp) in the sample; (c) comparing the amount to a reference; and (d) determining whether the sample has the following changes relative to the reference (i) an increased fungal infection; (ii) a decreased expression of Tyr; and/or (iii) a decreased expression of Tyrp.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C: Liver copper levels cannot be predicted by (A) serum copper levels, or (B) coat score. Green shaded animals were used for control arrays; red shaded animals were used for copper deficient arrays. (C) Liver copper levels correlate moderately well with liver selenium levels.

FIGS. 2A-2C: Gene expression arrays found (A) copper deficient animals are well separated from control animals by principal components analysis in both skin and blood. (B) Skin and blood have both distinct and overlapping sets of probes altered comparing copper deficient to control arrays. (C) Heat map of genes changed in copper deficient animals demonstrates that most of the genes changed in only skin or blood, not both, are not expressed at all in the other tissue type. Darker colors indicate higher expression levels.

FIGS. 3A-3E: Gene ontology groups differentially regulated in copper deficient animals include (A) coat color regulation, (B) adhesion and matrix, (D) blood vessel integrity, and (E) energy metabolism genes. Expression levels are relative to control pools. (C) Overview of gene ontology groups of 373 genes dysregulated in copper-deficient ear skin samples. Angular width corresponds to number of genes, while radius corresponds to statistical significance. Groups overlap because many genes fall into more than one ontology group; overlap is approximate. The inner white circle corresponds to p=0.05, the outer white circle to p=0.01

FIGS. 4A-4B: (A) Quantitative RT-PCR confirms alteration in cattle genes most dysregulated in blood from copper deficient animals. (B) Fungal genes accidentally included in cattle arrays correlate strongly with copper levels in both skin and blood samples.

FIGS. 5A-5F: As for the training herd, in the validation herd liver copper levels do not correlate with (A) serum copper or (B) coat score. (C) Also like the training herd, in the validation herd liver selenium correlates to liver copper. (D,E,F) In the validation herd, liver copper is weakly correlated with serum selenium (D), cobalt (E), and zinc (F), probably indicative of a supplementation effect.

FIGS. 6A-6D: Some genes that correlate with copper in the training cohort correlate with other minerals in the validation cohort. (A) Training cohort expression of Megf9 (triangles) and Prok2 (circles) correlate with liver copper levels with coefficients of −0.58 and −0.68 respectively, and p-values of 0.029 and 0.007 by correlation z-test respectively. Each symbol corresponds to measurements from one animal. (B) Megf9 (triangles) and Prok2 (circles) did not correlate with liver copper levels in the validation cohort. (C) Megf9 (triangles) and Prok2 (circles) correlate with liver zinc levels with coefficients of 0.64 and 0.78 respectively, and p-values of 0.013 and 0.001 by correlation z-test respectively. (D) A linear regression, combining Megf9, Prok2, and Nr4a3 expression, predicts liver zinc levels with a correlation Of 0.95 and p<0.0001 by correlation z-test.

FIGS. 7A-7C: Some genes that correlate with copper in the training cohort correlate with coat score in the validation cohort. (A) Training cohort expression of Icam (triangles) and Sele (circles) correlate with liver copper levels with coefficients of 0.81 and 0.86 respectively, and p-values of 0.0008 and 0.0001 by correlation z-test respectively. Each symbol corresponds to measurements from one animal. (B) These genes no longer correlate with liver copper levels in the validation cohort (symbols as above). (C) Both Icam (triangles) and Sele (circles) correlate to hair coat score with coefficients of −0.59 and 0.−52 respectively, and p-values of 0.03 and 0.05 by correlation z-test respectively.

FIGS. 8A-8C: Coat color genes Tyr and Tyrp correlate well to liver copper levels in both the training and validation cohorts. (A) Training cohort expression of Tyr (triangles) and Tyrp (circles) correlate with liver copper levels with coefficients of 0.66 and 0.75 respectively, and p-values of 0.01 and 0.003 by correlation z-test respectively. Each symbol corresponds to measurements from one animal. (B) Tyr (triangles) and Tyrp (circles) also correlate well with liver copper levels in the validation cohort, with coefficients of 0.62 and 0.59 respectively, and p-values of 0.03 and 0.04. (C) When Tyr and Tyrp expression are combined in a linear regression, they predict liver copper levels with a correlation of 0.76, p=0.004. Each symbol is the combined Tyr/Tyrp metric from one animal.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods and kits for predicting and reducing risk of copper deficiency in a ruminant subject or a ruminant herd. The unique methods disclosed herein allow use of samples such as blood, serum, or skin, which are far less invasive than samples obtained by liver biopsy, which is the current gold-standard for determining copper deficiency. The methods disclosed herein also do not require administering metal salts.

As used herein, a “ruminant subject” refers to an animal from the taxonomical suborder Ruminanti, which has a four-part stomach, including a rumen, reticulum, omasum, and abomasum. A ruminant subject is acquires nutrients from plant-based food by fermentation prior to digestion, principally through bacterial actions. The process typically requires the fermented ingesta to be regurgitated and chewed again. Examples of ruminant subject include, but are not limited to, cattle, goats, sheep, giraffes, yaks, deer, antelope.

In some embodiments of the presently-disclosed subject matter, a method for predicting copper deficiency or reducing risk of copper deficiency in a ruminant subject or a ruminant herd is provided, which involves providing a sample (e.g., blood, serum, skin) from the subject or at least one subject in the herd.

In some embodiments, the “sample” or “biological sample” can be blood, serum, or skin. It is also noted that in some embodiments the term sample or biological sample can refer to a sample from an individual subject for whom a prediction of copper deficiency or a reduction of risk of copper deficiency is performed. In other embodiments, the term sample or biological sample can refer to sample from another subject within a herd or a plurality of samples from subjects within the herd (e.g., an average of amounts or levels from multiple samples). In this regard, it is contemplated that results of the methods disclosed herein that are associated with a subject in a herd, a subset of subjects in a herd, or all subjects in a herd can be applied to any one or more subjects in the herd.

For example, such results of the methods disclosed herein could be applied to predict copper deficiency or to reduce risk of copper deficiency in a subject that is a member of a herd, regardless of whether a sample from that particular subject has been tested. In this regard, an entire herd can be assessed without requiring the testing of a sample from each and every member of the herd. For example, in some embodiments samples from about 10, 20, 30, 40, 50, 60, 70, or 80% of the herd could be provided.

In some embodiments, a method for predicting copper deficiency in a ruminant subject or a ruminant herd involves providing a sample from the subject or at least one subject in the herd, and measuring an amount of fungal infection in the sample; expression of tyrosinase (Tyr) in the sample, and/or expression of tyrosinase-related protein (Tyrp) in the sample. The measured amounts are compared to a reference, and the subject or herd is identified as having an increased risk of copper deficiency when, relative to the reference, the sample has an increased fungal infection; a decreased expression of Tyr; and/or a decreased expression of Tyrp.

Measuring expression of Tyr and/or Tyrp can be conducted by methods known to those skilled in the art is inclusive of measuring mRNA and/or protein. In some embodiments a probe that specifically binds to the Tyr or Tyrp can be used. In some embodiments, primer pairs can be used in a PCR-based measurement.

In embodiments that involve measuring an amount of fungal infection (fungal load), the measurement can be of an amount of Coccidioides fungal infection. Various methods for measuring fungal load are known in the art. In some embodiments, a fungal protein can be measured to make a determination of the amount of fungal infection. In some embodiments, the method can include measuring the expression of LOC100337426.

In some embodiments, a method for predicting copper deficiency can also include measuring the activity of Tyr in the sample. In this regard, it is anticipated that in some embodiments both expression of and activity (enzymatic activity) of Tyr in the sample can be measured, where a decrease in expression relative to the reference can be associated with an increased risk of coppery deficiency. Assays appropriate for assessing enzymatic activity of Tyr are known in the art. In some embodiments, additional markers can be measured in the sample, such as selenium, cobalt, zinc, and/or those set forth and incorporated herein.

The term “reference” as used herein can include, for example, an amount or level of a marker (e.g., fungal infection, LOC100337426, Tyr, Tyr activity, Tyrp, selenium, cobalt, zinc, another marker as set forth and incorporated herein) in one or more samples from one or more individuals without a copper deficiency. In some embodiments, the reference includes an amount or level of a marker in a sample from the subject(s) collected prior to initiation of treatment for the disease and/or onset of the disease and the biological sample is collected after initiation of the treatment or onset of the disease. In this regard, the methods disclosed herein can be used to reassess a subject or herd at a future date and/or after administration with a predicted effective copper supplementation dose.

As used herein, the terms “treatment” or “treating” relate to any treatment of a copper deficiency and include, for example, ameliorating or relieving the symptoms of a copper deficiency. As will be understood by those of ordinary skill in the art, when the term “prevent” or “prevention” is used in connection with a prophylactic treatment, it should not be understood as an absolute term that would preclude any sign of copper deficiency in a subject. Rather, as used in the context of prophylactic treatment, the term “prevent” can refer to inhibiting the development of a copper deficiency, limiting the severity of the coppery deficiency, arresting the development of the copper deficiency, and the like.

In some embodiments, the reference can include a standard sample. Such a standard sample can be a reference that provides amounts or at levels considered to be control amounts or levels. For example, a standard sample can be prepared to mimic the amounts or levels of one or markers associated with an absence of copper deficiency.

In some embodiments, the reference can include control data. Control data, when used as a reference, can comprise compilations of data, such as may be contained in a table, chart, graph, e.g., standard curve, or database, which provides amounts or levels of one or more markers considered to be control levels. Such data can be compiled, for example, by obtaining amounts or levels of one or more markers in one or more samples (e.g., an average of amounts or levels from multiple samples) from one or more individuals without the copper deficiency. The samples can be obtained from one or more individuals from a relevant geographic area, relevant species, etc.

The presently-disclosed subject matter also includes a method for reducing risk of copper deficiency in a ruminant subject or a ruminant herd, which also involves providing a sample from the subject or at least one subject in the herd, and measuring an amount of fungal infection in the sample, expression of tyrosinase (Tyr) in the sample, and/or expression of tyrosinase-related protein (Tyrp) in the sample. The method also involves estimating the level of copper deficiency in the subject or herd by correlating the measured amount to a reference; and calibrating a predicted effective copper supplementation dose based on the estimated level of coppery deficiency in the subject or herd. In this regard, a copper supplementation can be provided that is predicted to treat copper deficiency while limiting the risk of metal poisoning. Following such administration, a subject or herd can be reassessed by using newly-provided additional sample(s). It is further contemplated that in some embodiments, the subject(s) can also be inoculated with a fungus or fungal isolate.

The presently disclosed subject matter also includes kits useful for predicting copper deficiency in or reducing risk of copper deficiency in a ruminant subject or a ruminant herd, which include a probe or primer pair for each of at least two or more of Tyr, Tyrp, and LOC100337426. In some embodiments, the kits can also include a probe or primer pair for each of at least one marker as set forth and incorporated herein.

The presently disclosed subject matter also includes methods for predicting copper deficiency in or reducing risk of copper deficiency in a ruminant subject or a ruminant herd that involve receiving a dataset, the dataset comprising measured amounts of (i) fungal infection in a sample from the subject or at least one subject in the herd, (ii) expression of tyrosinase (Tyr) in the sample, and/or (iii) expression of tyrosinase-related protein (Tyrp) in the sample; and optionally further comprising measured amounts of (iv) selenium, cobalt, and/or zinc in the sample, and/or (v) at least one marker as set forth in and incorporated herein; and (b) processing the dataset, where processing comprises (i) calculating a risk index, wherein the calculating comprises: applying predetermined weights to the measured amounts; and solving one or more nonlinear differential equations, wherein the one or more nonlinear differential equations are based, at least in part, on the weights for the measured amounts; and (ii) correlating a risk index (e.g., AUC value) to an odds ratio, wherein the correlating comprises solving a logit probability model.

The presently-disclosed subject matter also includes systems for predicting copper deficiency in or reducing risk of copper deficiency in a ruminant subject or a ruminant herd, which includes a data access device configured to receive measured amounts of (i) fungal infection in a sample from the subject or at least one subject in the herd, (ii) expression of tyrosinase (Tyr) in the sample, and/or (iii) expression of tyrosinase-related protein (Tyrp) in the sample; and optionally measured amounts of (iv) selenium, cobalt, and/or zinc in the sample, and/or (v) at least one marker as set forth and incorporated herein; and a server in data communication with the data access device configured to perform: (i) calculating a risk index, wherein the calculating comprises: applying a predetermined weight to the measured amounts; and solving one or more nonlinear differential equations, wherein the one or more nonlinear differential equations is based, at least in part, on the weights for the measured amounts; and (ii) correlating the risk index to an odds ratio, wherein the correlating comprises solving a logit probability model.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

The presently-disclosed subject matter is further illustrated by the following specific but non-limiting examples. The following examples may include compilations of data that are representative of data gathered at various times during the course of development and experimentation related to the present invention.

### EXAMPLES

The studies described in this section relate to development and testing of an exemplary non-invasive or low-invasive method for assessing copper deficiency. Examples herein are directed to use gene expression arrays, and biological samples including skin and blood from copper deficient and control animals. Determination of whether the animals were copper deficient or control was made using the current gold-standard—liver biopsy.

Liver Copper Levels Do Not Correlate with Serum Copper Levels and Coat Scores.

In order to develop a non-invasive or low invasive test for copper deficiency, the present inventors assessed 15 cows from a herd previously known to have problems with copper deficiency. Coat scores were assessed, liver biopsies taken, blood drawn, and skin samples collected by ear punch. Liver and blood samples were sent to a commercial testing lab for levels of copper, cobalt, iron, manganese, molybdenum, selenium, and zinc (Table 1). RNA was isolated from whole homogenized skin and blood samples.

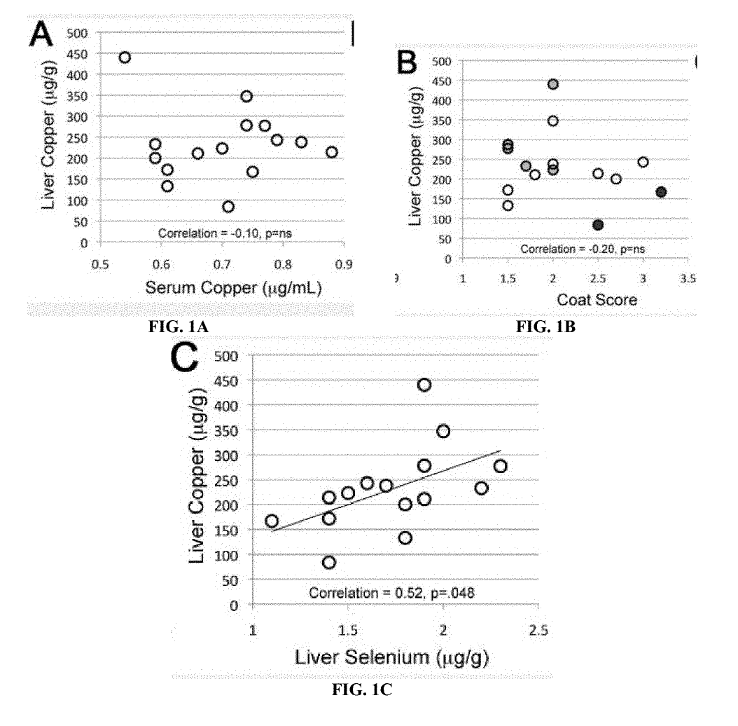

Copper deficiency in these animals was at worst moderate, but liver copper levels were not significantly correlated with either serum copper levels or coat scores (FIG. 1A, 1B). Liver copper levels did not correlate with serum levels of any of the other metals tested (not shown), but was significantly correlated with liver selenium levels (FIG. 1C).

Gene Expression Changes Associated with Copper Deficiency are Detectable in Skin and Blood.

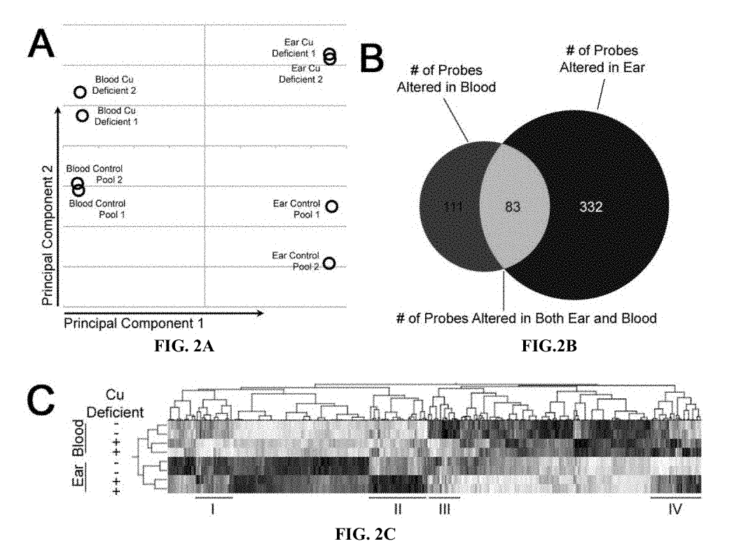

Samples were selected for expression array analysis based on a combination of coat scores, liver copper levels, and high quality RNA in both ear (skin) and blood samples; samples selected are marked with red and green coloration in FIG. 1B. Samples were applied to Affymetrix Bovine Genome 1.0 arrays. Arrays were normalized in a group using Affymetrix software, and RNA output used for further analysis. Low expression genes were filtered to reduce noise. Of the 26,773 probe sets on the array, there were 13266 named probe sets with expression >=7 average in any group. Principle Components Analysis was applied to these 13266 probe sets across the eight arrays. As expected, most of the variability in expression (92%) was attributable to the difference between blood and skin, but of the remaining variability, the majority of the variability was attributable to a component that separated the copper deficient animals from the control animals (FIG. 2A). This principle component explained 56% of the variability in the ear samples and 48% of the variability in the blood samples. Given small numbers of free-living, outbred animals, this is a very strong signal.

The strength of the copper-deficiency-related changes was higher in the skin samples than in the blood samples. In blood, there were 194 probe sets with p<0.05 of greater than 20% change in expression, while in skin with the same conditions there were 415, including congruent changes in 83 probe sets of the 194 (43%) (FIG. 2B).

A heat map with hierarchical clustering of these combined 526 probe sets illustrates several salient points about this data (FIG. 2C). First, only ˜16% of the genes have congruent changes (primarily in sections I and IV, as indicated by bars under the heat map). However, even when the changes are congruent, they may be from a different baseline. For instance, in Section I, copper deficiency results in down regulation of these genes in both blood and skin, but from a much higher expression level in skin. Another relevant point is that many of the strongest genes in each tissue type are not replicated in the other. For instance, section II consists of genes strongly upregulated by copper deficiency in the ear skin, but barely expressed at all in blood. Section III shows genes strongly downregulated by copper deficiency in blood, but with no consistent change in skin.

### Even Moderate Copper Deficiency Impacts Genes Related to Vascular Integrity in Skin.

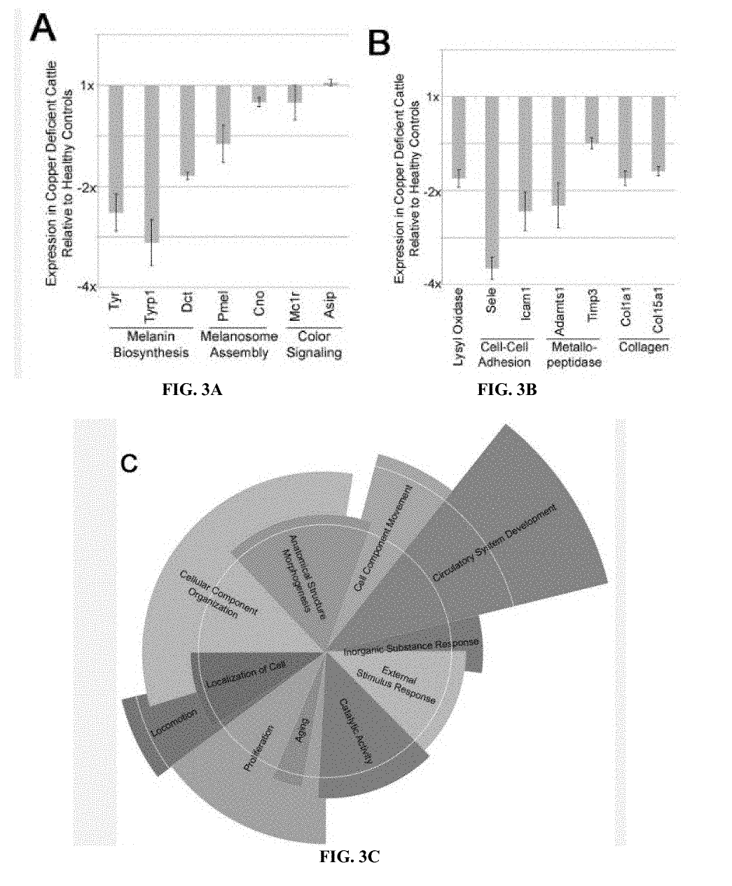

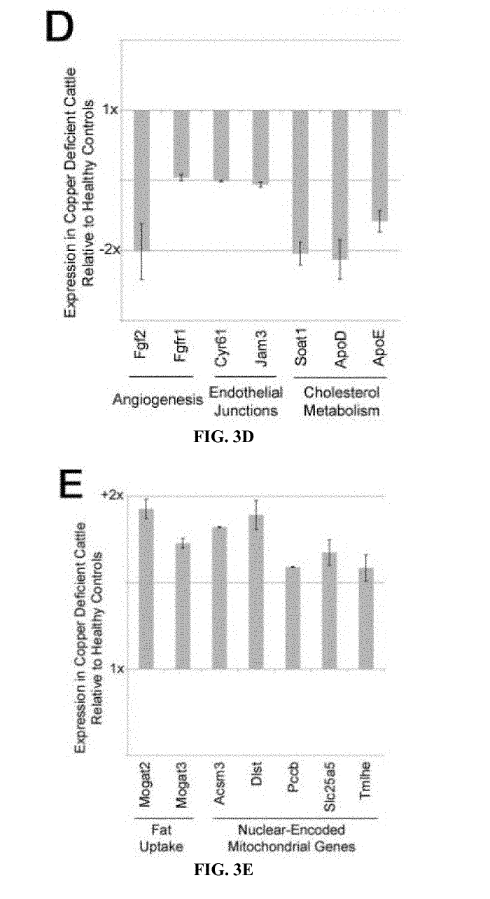

Copper deficiency is known to result in functional suppression of tyrosinase activity in melanocytes, resulting in characteristic coat color changes in cattle, and in reduced lysyl oxidase activity, resulting in elastin and cell-cell adhesion difficulties. The present inventors found that at the level of gene expression, there were alterations in regulation of these pathways in copper deficient cattle. Copper deficiency resulted in suppression of melanin biosynthesis related genes including tyrosinase, dopachrome tautomerase, and tyrosinase-related protein 1, but other coat-color signaling or melanosome assembly genes were less affected (FIG. 3A). Similarly, the copper-dependent protein lysyl oxidase is downregulated, as are genes related to its functions, including cell-cell adhesion, metallopeptidase, and collagens (FIG. 3B). Altogether, there are 21 differentially regulated extracellular matrix genes (not shown). None of these genes related to melanin or lysyl oxidase are differentially regulated in blood.

Next, we used an undirected approach to identify statistically overrepresented gene ontology groups in genes dysregulated in copper-deficient ear skin samples. The 415 probe sets corresponded to 373 unique Entrez IDs (many of the probe sets were uncharacterized open reading frames). The majority of these fell into statistically overrepresented gene ontology groups, including a large number of groups associated with cellular or cellular component movement, proliferation, stimulus response, catalytic activity, and circulatory system development (FIG. 3C). Angular width corresponds to number of genes, while radius corresponds to statistical significance. The simplest explanation for these data is that the effect of copper deficiency on vascular integrity is earlier and more dramatic in its effects than previously suspected, resulting in failure of genes needed to maintain vascular integrity (FIG. 3D). These changes likely come at a cost of increased energy use (and thus decreased performance), suggested by the increase in expression of fat uptake and nuclear mitochondrial genes (FIG. 3E).

Gene Expression Changes in Whole Blood RNA suggest increased Fungal Infection.

The 194 probe sets with altered regulation with copper deficiency in RNA from whole blood correspond to 162 unique entrez IDs. Ontology groups with altered regulation include 87 in the metabolic process category, 54 in stimulus response, 11 microtubule-based process, 11 (all downregulated) M phase of mitotic cell cycle, and 20 cytoskeletal, among others (some genes may be in more than one group). However, none of these groups are statistically overrepresented, and they do not form as coherent a picture as the dysregulated genes in skin.

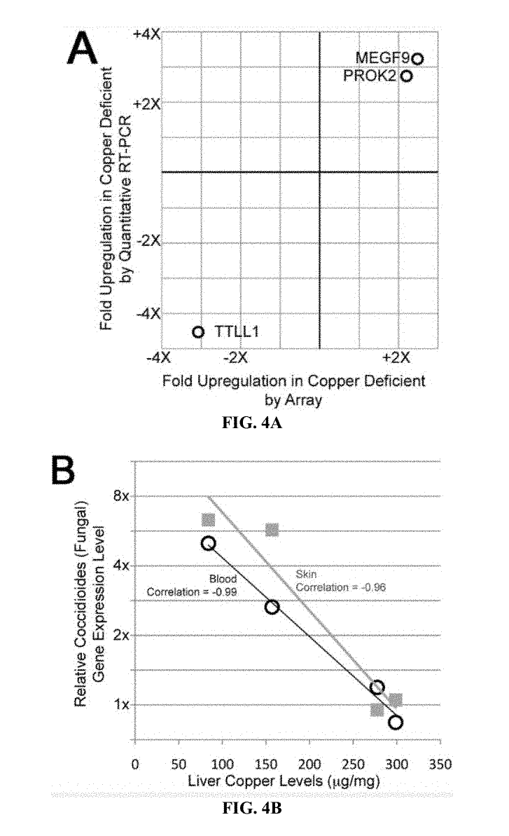

Part of the difficulty in forming a coherent picture, though, may lie in the fact that many of the genes that are most strongly dysregulated are uncharacterized: of the 10 probe sets with the highest dysregulation in whole blood RNA, 7 have unknown function. The three with known function include tubulin tyrosine ligase (Tt111), previously shown to be regulated in copper toxicity, prokineticin 2 (Prok2), related to myeloid cell mobilization from the bone marrow, and Megf9, a transmembrane protein with similarities to the laminin family of structural scaffolding proteins. Expression levels of Tt111, Prok2, and Megf9 were probed by quantitative RT-PCR and found to perfectly reproduce the alterations in expression levels predicted by the arrays (FIG. 4A). The 7 uncharacterized genes include two uncharacterized bovine microRNA, MIR2285a and MIR2286, and five genes predicted based on either bovine ESTs, prediction algorithms, or both. Some of these are similar to known proteins—for instance, L00512150 is similar to a myeloid differentiation marker. However, on inspection we found that one of these, LOC100337426, was a perfect match to a protein from the fungal strain Coccidioides. Coccidioides infection is endemic in cattle, but generally harmless, and it is likely that parts of the Coccidioides genome was accidentally included in the bovine genome sequencing project, and because it was included in the bovine genome project, was included on the Affymetrix array. Coccidioides has been known to infect human subjects, and in the era before improved anti-fungal drugs were discovered, was treated with colloidal copper (Jacobson HP 1927). Copper deficiency thus allows an increase in fungal infection (FIG. 4B).

### Mineral Levels in Validation Cohort Serum and Liver.

In order to validate the results obtained in our training herd, the property of a private owner in northern Tennessee, we collected a second set of samples from 15 cattle in a validation cohort, part of the Beef Unit at Middle Tennessee State University. As before, coat scores were assessed, liver biopsies taken, blood drawn, and skin samples collected by ear punch. Liver and blood samples were sent to a commercial testing lab for levels of copper, cobalt, iron, manganese, molybdenum, selenium, and zinc (Table 2). None of these animals had clinical copper deficiency; the range is from 106 μg/g to 292 μg/g (Table RNA was isolated from whole homogenized skin and blood samples.

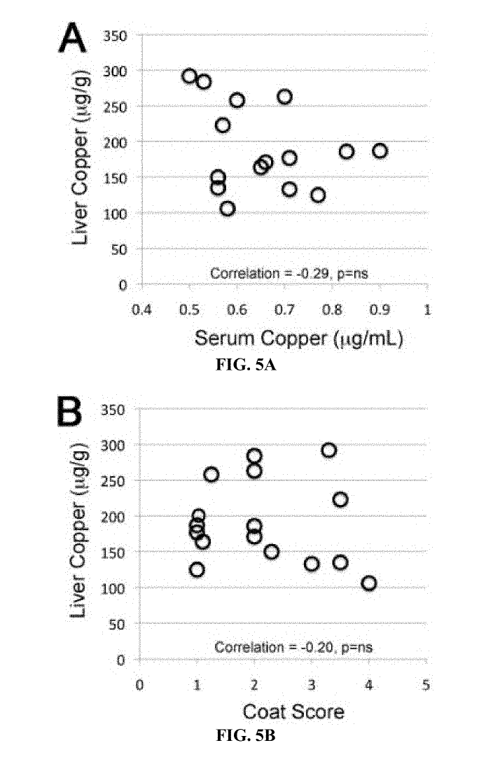

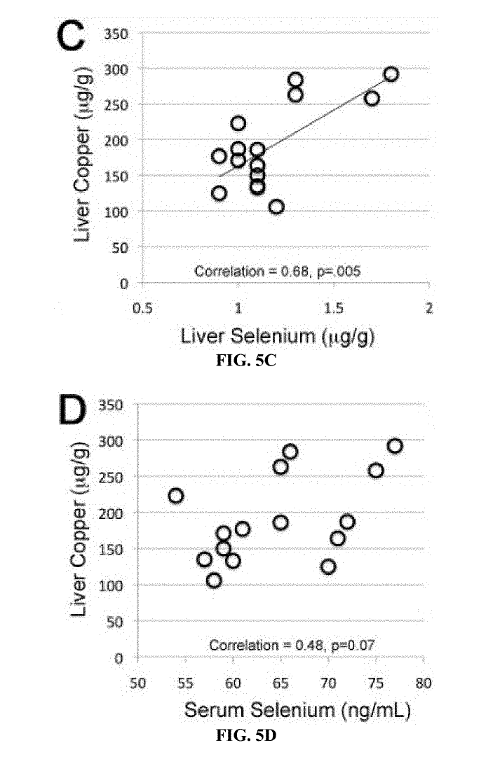

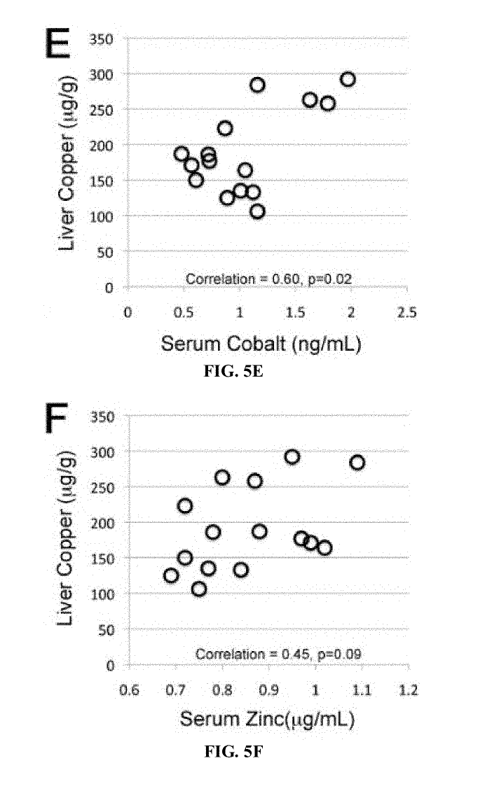

As before, liver copper levels did not correlate with either serum copper (FIG. 5A) or with coat scores (FIG. 5B), but did correlate with liver selenium (FIG. 5C). Unlike the previous cohort, however, liver copper levels did correlate with moderate significance and strength to serum selenium (FIG. 5D), cobalt (FIG. 5E), and zinc (FIG. 5F). Because there have been no prior reports of correlations in these measures, this is likely to be a supplementation effect; overall mineral levels are higher in cattle with greater supplement consumption or uptake.

### Validation of Markers Identified in the Training Herd.

Quantitative RT-PCR was used to measure expression of genes correlated with liver copper levels in the training herd. We found that in general, genes correlated with liver copper levels in the training herd fell into three classes in the validation herd: (I) Genes that correlated with a different mineral besides copper, (II) Genes that correlated with coat scores, but not copper, and (III) Genes that correlated with copper in both training and validation cohorts.

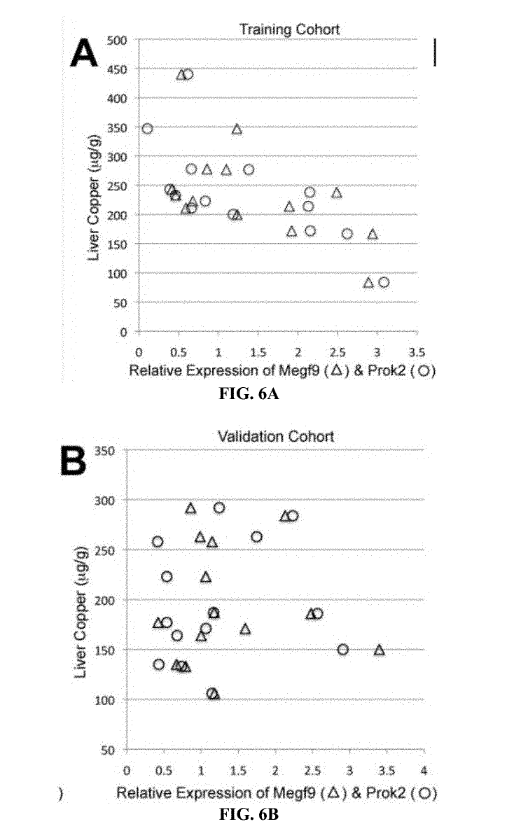

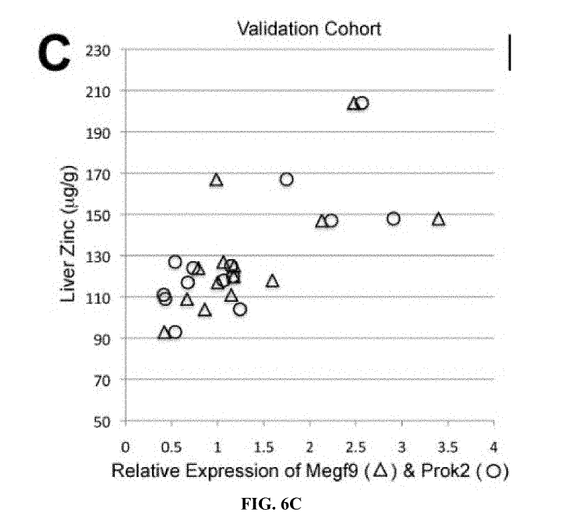

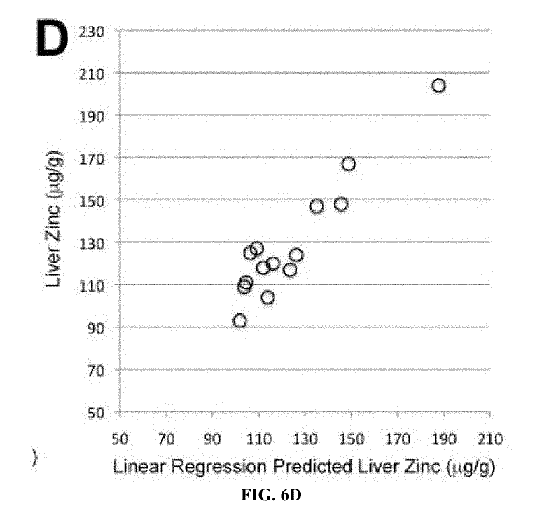

As an example of Group I, genes that correlated well with copper in the training cohort but with other minerals in the validation, consider Prok2 and Megf9. In the overall set of 15, these correlated with liver copper with coefficients of −0.58 and −0.69 respectively (FIG. 6A). They also correlate with each-other with a coefficient of 0.92. However, in the validation cohort, they were statistically uncorrelated with copper levels, with correlation coefficients near zero (FIG. 6B). These genes were, however, strongly positively correlated with liver zinc levels (FIG. 6C), and a linear regression combining these two genes with a third, NR4A3, matches liver zinc levels with a correlation of 0.95 (FIG. 6D). Even with a multiple testing correction (we tested levels of 7 minerals in the liver), the p value is less than 0.0001. It thus seems likely that there is a category of genes, including these, whose expression is related to mineral levels more broadly, rather than being specific to copper (or zinc).

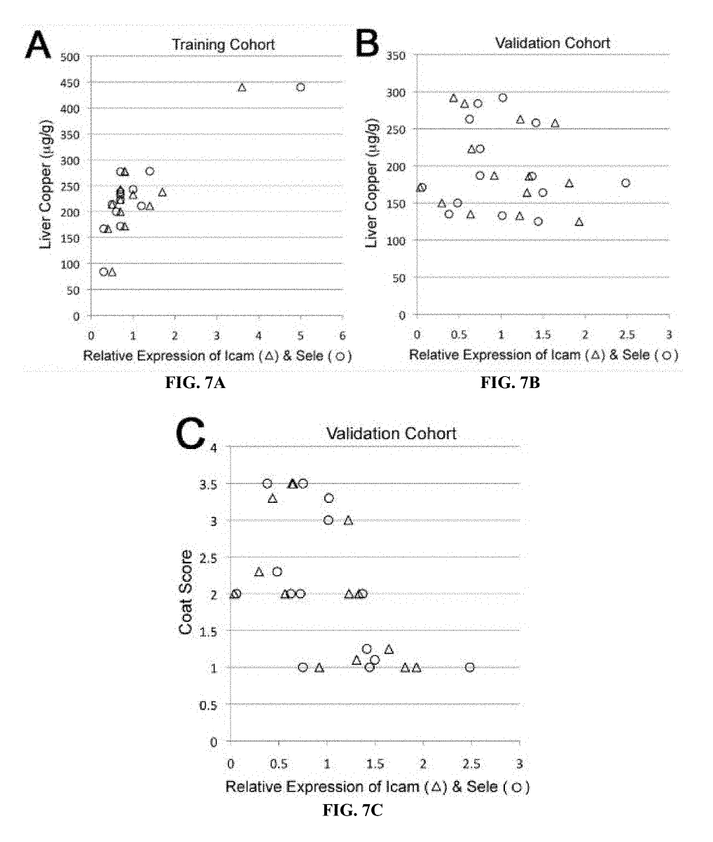

As an example of Group II, genes that correlated well with copper in the training cohort but with coat scores in the validation cohort, consider genes related to cell-cell adhesion and vascular integrity, ICAM and SELE (array results in FIG. 3B). By quantitative RT-PCR on all of the cattle in the training set, these correlated to liver copper levels with coefficients of 0.81 and 0.86 respectively (FIG. 7A). Even if we drop the high outlier, correlations remain moderately strong, of 0.38 and 0.68 respectively (and p value for SELE is still 0.02 without the high outlier). These genes no longer correlate with liver copper levels in the validation cohort (FIG. 7B). However, they do correlate with coat score (FIG. 7C), with higher expressions of Icam and Sele usually corresponding to lower (better) coat scores. This association between expression of these genes and coat score was also present, although more weakly, in the training cohort, with a correlation of −0.48 each, rising to 0.56 and p=0.05 with linear regression).

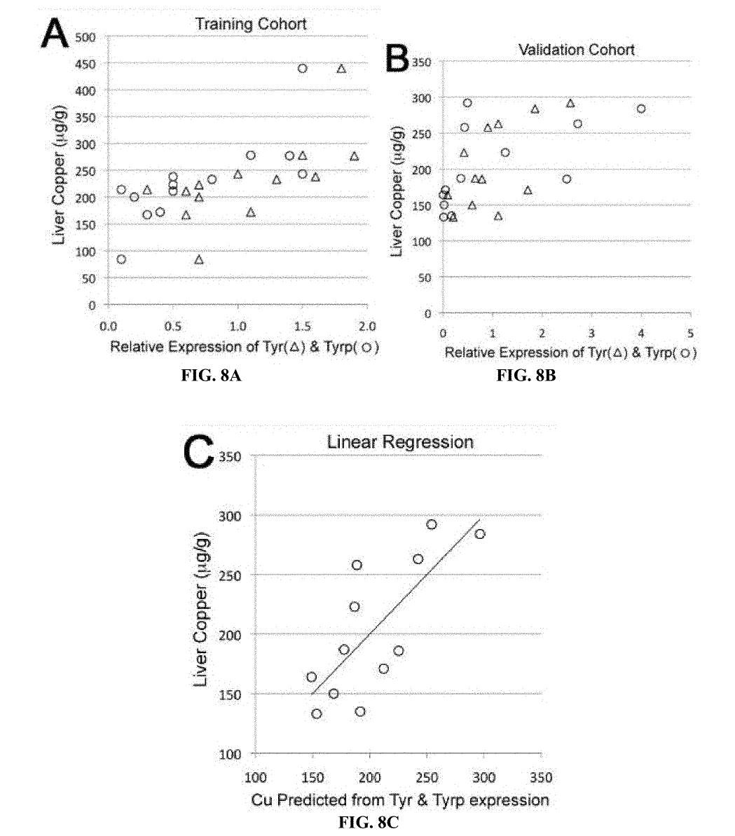

Finally, there exist a group of genes which correlated well with liver copper levels in both the training and validation cohort (Group III). As an example of this, consider the coat color genes Tyr, and Tyrp. In the training cohort, expression of these correlated to liver copper levels with coefficients of 0.66 and 0.75 respectively (FIG. 8A). This was matched in the validation set, in which Tyr and Tyrp correlated to liver copper with coefficients of 0.62 and 0.59 (FIG. 8B); when combined in a linear regression, this was strengthened to a correlation of 0.76 (FIG. 8C).

All publications, patents, and patent applications mentioned in this specification, and including those set forth in the following list, are herein incorporated by reference to the same extent as if each individual publication, patent, or patent application was specifically and individually indicated to be incorporated by reference:

### REFERENCES

1. U.S. Patent Application Publication No. 2008/0090297 of Richards for “Using metal responsive biomarker as evaluative tool in determining relative zinc or copper bioavailability in animals.”
2.Davis G K, Mertz W. Copper. In: Mertz W, editor. Trace elements in human and animal nutrition, vol I. San Diego, New York, Berkeley, Boston, London, Sydney, Tokyo, Toronto: Academic Press, 1987:301-350.
Linder M C. Biochemistry of copper. In: Frieden E. Ser., editor. Biochemistry of the elements, vol. 10. New York, London: Plenum Press, 1991.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating copper deficiency in a ruminant subject or a ruminant herd, comprising:
(a) providing a sample from the subject or at least one subject in the herd;
(b) measuring an amount of
(i) fungal infection in the sample;
(ii) expression of tyrosinase (Tyr) in the sample, and/or
(iii) expression of tyrosinase-related protein (Tyrp) in the sample;
(c) estimating the level of copper deficiency in the subject or herd by correlating the measured amount to a reference;
(d) calibrating a predicted effective copper supplementation dose based on the estimated level of coppery deficiency in the subject or herd; and
(e) administering the predicted effective copper supplementation dose to the subject or at least one subject in the herd and/or inoculating the subject or at least one subject in the herd with a fungus or fungal isolate.

2. The method of claim 1, and further comprising providing a second sample from the subject or at least one subject in the herd that has been administered the effective supplementation, recalibrating a predicted effective copper supplementation dose based on the estimated level of coppery deficiency in the subject or herd using the second sample.

3. The method of claim 1, and further comprising measuring the activity of Tyr in the sample.

4. The method of claim 1, wherein measuring the amount of fungal infection comprises measuring the amount of Coccidioides fungal infection.

5. The method of claim 1, and further comprising measuring the expression of LOC100337426.

6. The method of claim 1, and further comprising measuring the amounts of selenium, cobalt, and/or zinc in the sample.

7. The method of claim 1, and further comprising contacting the sample with a probe for Tyr and/or Tyrp.

8. The method of claim 1, and further comprising providing primer pairs for use in measuring the expression of Tyr and/or Tyrp.

9. A method for predicting copper deficiency in and treating copper deficiency in a ruminant subject or a ruminant herd, comprising:
(a) receiving a dataset, the dataset comprising measured amounts of
(i) fungal infection in a sample from the subject or at least one subject in the herd,
(ii) expression of tyrosinase (Tyr) in the sample, and/or
(iii) expression of tyrosinase-related protein (Tyrp) in the sample;
and optionally further comprising measured amounts of
(iv) selenium, cobalt, and/or zinc in the sample; and
(b) processing the dataset, where processing comprises
(i) calculating a risk index, wherein the calculating comprises:
applying predetermined weights to the measured amounts; and
solving one or more nonlinear differential equations, wherein the one or more nonlinear differential equations are based, at least in part, on the weights for the measured amounts; and
(ii) correlating a risk index to an odds ratio, wherein the correlating comprises solving a logit probability model.

10. A system for predicting copper deficiency in a ruminant subject or a ruminant herd, comprising:
(a) a data access device configured to receive measured amounts of fungal infection in a sample from the subject or at least one subject in the herd,
(ii) expression of tyrosinase (Tyr) in the sample, and/or
(iii) expression of tyrosinase-related protein (Tyrp) in the sample; and optionally measured amounts of
(iv) selenium, cobalt, and/or zinc in the sample; and
(b) a server in data communication with the data access device configured to perform:
(i) calculating a risk index, wherein the calculating comprises:
applying a predetermined weight to the measured amounts; and
solving one or more nonlinear differential equations, wherein the one or more nonlinear differential equations is based, at least in part, on the weights for the measured amounts; and
(ii) correlating the risk index to an odds ratio, wherein the correlating comprises solving a logit probability model.

11. A method for identifying fungal infection and expression of tyrosinase and tyrosinase-related protein in a ruminant subject or a ruminant herd, comprising:
(a) providing a sample from the subject or at least one subject in the herd;
(b) measuring an amount of
(i) fungal infection in the sample;
(ii) expression of tyrosinase (Tyr) in the sample, and/or
(iii) expression of tyrosinase-related protein (Tyrp) in the sample;
(c) comparing the amount to a reference; and
(d) determining whether the sample has the following changes relative to the reference
(i) an increased fungal infection;
(ii) a decreased expression of Tyr; and/or
(iii) a decreased expression of Tyrp.

12. The method of claim 11, and further comprising measuring the activity of Tyr in the sample.

13. The method of claim 11, wherein measuring the fungal infection comprises measuring the Coccidioides fungal infection.

14. The method of claim 11, and further comprising measuring the expression of LOC100337426.

15. The method claim 11, and further comprising measuring the amounts of selenium, cobalt, and/or zinc in the sample.

16. The method of claim 11, and further comprising contacting the sample with a probe for Tyr and/or Tyrp.

17. The method of claim 11, and further comprising providing primer pairs for use in measuring the expression of Tyr and/or Tyrp.

18. The method of claim 11, wherein the sample is a blood, serum, or skin sample.

19. The method of claim 11, and further comprising administering a copper supplementation dose to the subject or at least one subject in the herd and/or inoculating the subject or at least one subject in the herd with a fungus or fungal isolate when the sample includes an increased fungal infection; a decreased expression of Tyr; and/or a decreased expression of Tyrp.
